(19) United States
(12) Patent Application Publication
(10) Pub. No.: US 2018/0164274 A1
(43) Pub. Date: Jun. 14, 2018

(54) Emulsion Stability Measurement System and Method

(71) Applicants: John D. Norwood, Houston, TX (US); David Hoffman, Houston, TX (US)

(72) Inventors: John D. Norwood, Houston, TX (US); David Hoffman, Houston, TX (US)

(73) Assignee: OFI Testing Equipment, Inc., Houston, TX (US)

(21) Appl. No.: 15/836,248

(22) Filed: Dec. 8, 2017

Related U.S. Application Data
(60) Provisional application No. 62/433,458, filed on Dec. 13, 2016.
(60) Provisional application No. 62/520,355, filed on Jun. 15, 2017.

(51) Int. Cl. G01N 33/28; G01N 27/92

(52) U.S. Cl. CPC G01N 33/2823; E21B 2049/085; G01N 27/92

(57) ABSTRACT

Embodiments of an emulsion stability probe generally include two opposing electrodes with a gap there between, one or more electrical circuitry devices embedded in a probe assembly, and electrical circuitry devices that function to quantify and manipulate a sensed current signal, wherein the manipulated signal can be transmitted to an emulsion stability meter. Embodiments of an in-line emulsion stability measurement system generally include an electrical stability probe equipped with a process-connectable fitting, wherein attachment of the electrical stability probe to a process piping segment, utilizing the fitting, provides the electrodes and gap at least partially within the process piping segment, and whereby an in-line emulsion stability measurement of process fluid disposed within the piping segment may be performed. Methods of performing an emulsion stability measurement in-line and/or using an embodiment of the emulsion stability probe disclosed herein are also provided.

### CROSS-REFERENCE TO RELATED APPLICATIONS

This application claims the benefit of U.S. Provisional Application No. 62/433,458 filed on Dec. 13, 2016, and U.S. Provisional Application No. 62/520,355, filed on Jun. 15, 2017, which applications are incorporated herein by reference as if reproduced in full below.

### STATEMENT REGARDING FEDERALLY SPONSORED RESEARCH OR DEVELOPMENT

Not applicable.

### BACKGROUND OF THE INVENTION

### Field of the Invention

The present invention generally relates to an improved apparatus and method for measuring, including measuring in-line, the stability of fluids, such as emulsions, including but not limited to, oil-based drilling fluids.

### Description of the Related Art

In the oil and gas industry, it is necessary to understand the properties of materials used during subterranean exploration. One such class of materials is liquid emulsions formulated at the surface and then provided downhole. Included in the assortment of liquid emulsions utilized in downhole operations are drilling fluids (“muds”) comprising water-in-oil or invert emulsions. An industry desired property to be quantified for many such materials is emulsion stability.

The prior art includes apparatuses and methods for measuring the stability of drilling fluid emulsions. Examples include U.S. Pat. No. 6,906,535 to Murphy, Jr. et al.; U.S. Pat. No. 7,701,229 to Murphy et al.; U.S. Pat. No. 7,830,161 to Murphy; U.S. Pat. No. 8,994,389 to Growcock et al.; and U.S. Pat. No. 9,134,291 to Jamison et al. Each of these prior art patents is incorporated herein by reference in its entirety.

Typically, emulsion stability is measured by taking a process sample and employing a stand-alone emulsion stability meter device, such as provided in the Model #131-50 Emulsion Stability Tester (EST) available from OFI Testing Equipment, Inc. of Houston, Tex. The EST works by utilizing a probe immersed into a sample of the oil-based drilling fluid such that the fluid fills a gap between a pair of electrodes. An increasing AC voltage is applied to the oil-based drilling fluid sample, across the two electrodes, while the current flow between them is monitored. Once the potential difference between the electrodes reaches a certain level, the drilling fluid invert emulsion will break down and a conductive path will be formed between the two electrodes. The peak voltage required to cause the breakdown is defined as Electrical Stability of the oil-based drilling fluid. The EST signals, probe assembly electrode arrangement, and the testing procedure are specified in Section 11 of The American Petroleum Institute's “Recommended Practice Standard Procedure for Field Testing Oil-Based Drilling Fluids.” API Recommended Practice 13B-2, Fifth Edition, April 2014 (the “API Procedure”), which is incorporated herein by reference.

Of considerable importance is the accuracy of these emulsion stability measurements. Any phenomenon or circumstance that affects the accuracy of the emulsion stability measurement is undesirable. Typical factors which influence emulsion stability measurement accuracy include electrical signal interference, temperature fluctuations, probe cable length, and fluid contamination in an emulsion stability meter connector plug or socket. In addition, current EST design does not provide for full testing of the probe and cable after the API Procedure is performed.

It is an objective of the present invention therefore to provide an apparatus and method for measurement of emulsion stability which removes or reduces these negative testing influences.

Of primary importance is the observation of the trend of these emulsion stability measurements as a function of time. Any trending deviations from historical measurements may be indicative of significant (and undesired) changes in the chemical composition of the drilling fluid. Such deviations could be caused by water/oil influx or undesired increases/decreases in solids content.

As previously described, emulsion stability is typically measured by taking a process sample and introducing it to a stand-alone emulsion stability meter device. Accordingly, to measure emulsion stability changes over time, multiple process samples must be provided and multiple off-line emulsion stability measurements performed. Such repetitive process sampling and disposal or recycling of obtained process samples is cumbersome and inefficient. Handling of drilling fluid process samples is also potentially hazardous to personnel and, potentially, to the environment.

It is an objective of the present invention therefore to provide an apparatus and method for in-line measurement of emulsion stability, which obviates the need for obtaining, handling, and performing off-line measurements of process samples.

### BRIEF SUMMARY OF THE INVENTION

Embodiments of an apparatus of the invention generally include an EST probe comprising a molded polymeric material and having an electronic printed circuit board (PCB) embedded there within. In one embodiment, the probe comprises signal detection and conditioning circuitry to measure induced current (the “current sense signal”) directly at the probe and translate it into a form that can be sent down the cable to the measurement box without being distorted by the typical sources of EST probe errors. In certain embodiments, the probe provides for converting the current sense signal into a differential analog signal or digital signal. In various embodiments, the probe PCB includes temperature sensing and/or data storage functionality. In one embodiment, the probe comprises recessed terminals that allow test plugs to be connected directly to the end of the probe. Embodiments of a method of the invention generally include operation of an emulsion stability meter utilizing a probe of the present invention to measure the emulsion stability of a process fluid.

Further embodiments of an apparatus of the invention generally include an electrical stability probe, including but not limited to the improved EST probe disclosed herein, contained at least partially within a process-connectable fitting, wherein the electrical stability probe comprises two opposing electrodes separated by a gap and is in electronic communication, via a cable, with an emulsion stability meter. Embodiments of a method of the invention generally include reversible attachment of the fitting to a process piping segment, whereby the electrical stability probe is positioned at least partially within the process piping segment and is immersed in process fluid contained therein, whereupon operation of the emulsion stability meter provides an in-line emulsion stability measurement of the process fluid.

Other features and advantages of the invention will be apparent from the following description, the accompanying drawings, and the appended claims.

### BRIEF DESCRIPTION OF THE DRAWINGS

FIG. 1 is a depiction of an embodiment of an emulsion stability tester of the present invention.

FIG. 2 is a depiction of an embodiment of an emulsion stability probe of the present invention.

FIG. 3 is a listing of steps of an embodiment of a method of the present invention.

FIG. 4 is a depiction of an embodiment of an in-line emulsion stability measurement system of the present invention.

FIG. 5 is a side-view depiction of an embodiment of an electrical stability probe, contained within a fitting, of the present invention.

FIG. 6A is a depiction of an embodiment of an electrical stability probe, contained within a fitting, which is attached to a process piping segment, of the present invention.

FIG. 6B is a depiction from another angle of the embodiment of an electrical stability probe, contained within a fitting, which is attached to a process piping segment, of FIG. 6A.

FIG. 7 is listing steps of an embodiment of a method of the present invention.

### DETAILED DESCRIPTION OF EXEMPLARY EMBODIMENTS OF THE INVENTION

The exemplary embodiments are best understood by referring to the drawings wherein like reference characters designate like or similar parts throughout. As used herein, relative direction terms such as “top,” bottom,” “up,” and “down” are used only for descriptive purposes in illustrating exemplary embodiments, and other geometries and/or orientations are contemplated.

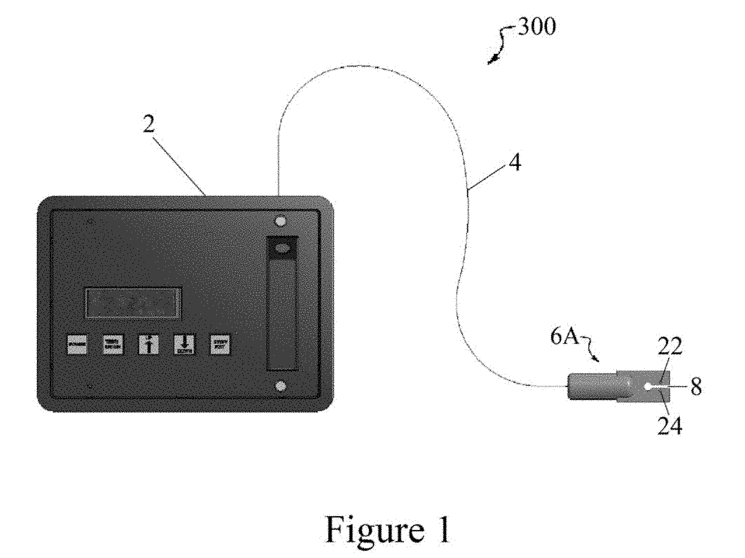

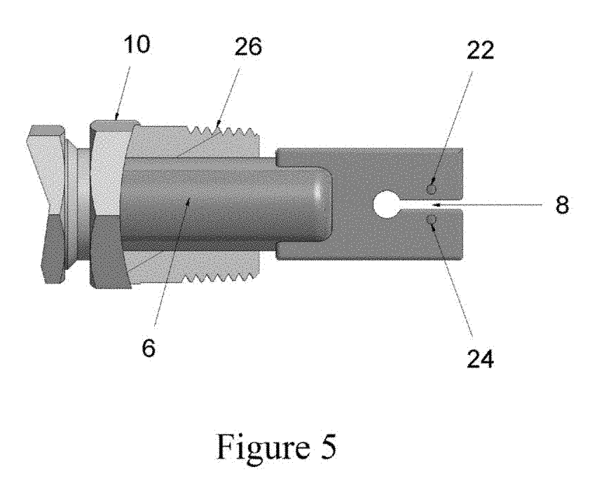

FIG. 1 depicts an embodiment of an emulsion stability measurement system (emulsion stability tester (EST)) 300 of the present invention. In one embodiment, emulsion stability measurement system 300 comprises an emulsion stability meter 2. In one embodiment, an emulsion stability meter 2 comprises a standard device, such as the above-mentioned emulsion stability meter available from OFI Testing Equipment, Inc. of Houston, Tex., or a similar device, as is known within the art. In one embodiment, the emulsion stability meter 2 may be in informational communication with one or more processing devices (not shown), such as a computer. Typically, an emulsion stability meter 2 is connected via a cable 4 to a standard electrical stability probe 6 (see FIG. 5), as is generally known within the art. In one aspect, connection of cable 4 to emulsion stability meter 2 utilizes a connection component (not shown), as is known within the art.

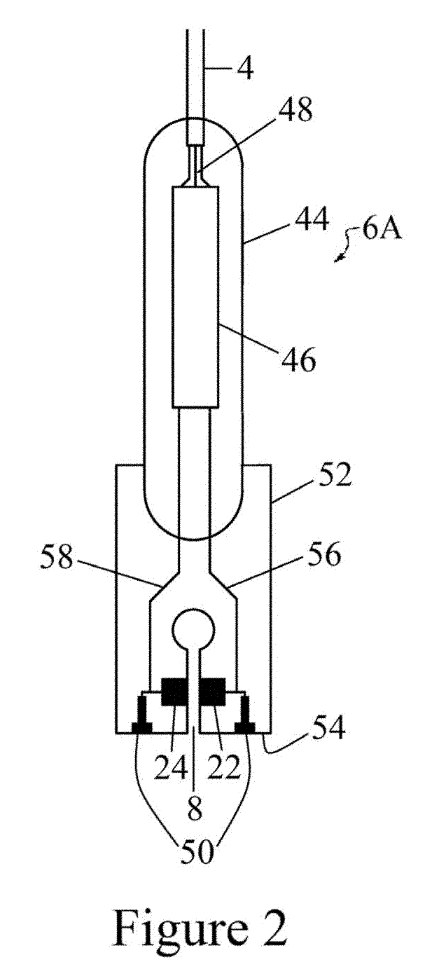

In one embodiment of the present invention, the emulsion stability meter 2 is connectable via cable 4 to an electrical stability probe 6A. In one embodiment (not shown), such connection is achieved without use of the aforementioned connection component (not shown), but rather via direct connection of cable 4 to one or more components (not shown) within emulsion stability meter 2, thereby removing any signal distortion/degradation attributable to the connection component (not shown). In one embodiment, cable 4 is adapted and configured to transmit electrical signals from emulsion stability meter 2 to electrical stability probe 6A and/or from stability electrical stability probe 6A to emulsion stability meter 2. In one embodiment, cable 4 is adapted and configured to transmit information from emulsion stability meter 2 to electrical stability probe 6A and/or from stability electrical stability probe 6A to emulsion stability meter 2. In one embodiment, electrical stability probe 6A comprises a pair of electrodes 22, 24 disposed in a probe tip 52 (shown in more detail in FIG. 2) with a probe gap 8 there between, as is well known within the art.

In one embodiment, stability electrical stability probe 6A is adapted to be at least partially immersed in a fluid sample (not shown) to be tested. In one aspect, the fluid sample (not shown) may be contained in a sample vessel (not shown), such as a thermostatically controlled viscometer cup. In one embodiment, as described below in detail, the fluid sample (not shown) may be contained within process piping. In one embodiment (not shown), an emulsion stability measurement system 300 comprises a temperature measurement device (not shown), electrically connected to emulsion stability meter 2, which is adapted and configured to measure temperature of the sample fluid (not shown). In one embodiment, a portion of the temperature measurement device (not shown) may be at least partially immersible in the fluid sample (not shown). In one embodiment (not shown), the temperature measurement device (not shown) may be electrically connected to emulsion stability meter 2 at least partially via cable 4.

FIG. 2 depicts an embodiment of an electrical stability probe 6A of the present invention. As shown in FIG. 2, electrical stability probe 6A comprises electrodes 22 and 24 separated by probe gap 8 and disposed within probe tip 52. In the embodiment of FIG. 2, electrical stability probe 6A comprises a probe assembly 44. In one embodiment, probe assembly 44 comprises a synthetic material, such as but not limited to, a polymeric epoxy material. In one embodiment, one or more electrical circuitry devices 46, such as a printed circuit board (PCB), are embedded within probe assembly 44. In one embodiment, an electrical circuitry device 46 may be in electronic and/or informational communication with one or more other electrical circuitry devices 46. In one embodiment, one or more cable 4 connection components 48, which provide communication informationally and/or electrically between cable 4 and at least one of the one or more electrical circuitry devices 46, are embedded within probe assembly 44. In one embodiment, electrodes 22, 24 are in electronic communication with at least one electrical circuitry device 46 via electrode wires 56, 58, respectively.

In one aspect, probe assembly 44 functions to physically protect at least electrical circuitry devices 46 and cable 4 connection components 48 from contamination by the fluid sample (not shown) and other external substances (not shown). In one aspect, probe assembly 44 functions to electrically insulate at least electrical circuitry devices 46 and cable 4 connection components 48 from external contact and possible electrical conduction with the test sample fluid, test vessel, and/or the user.

In one embodiment, an electrical circuitry device 46 comprises signal detection and conditioning circuitry to measure the current sense signal via electrodes 22, 24, and manipulate the current sense signal. In one embodiment, such signal manipulation comprises converting the electrical current sense signal to an electrical and/or informational form that can be transmitted to emulsion stability meter 2, via cable 4, with reduced signal distortion or degradation. Such form may include, but is not limited to, differential analog signals or digital signals. In one embodiment, an electrical circuitry device 46 comprises functionality to amplify and/or filter the electrical current sense signal, differential analog signals, and/or digital signals.

In one embodiment, an electrical circuitry device 46 may comprise functionality that includes, but is not limited to, temperature measurement and data storage. In one embodiment, such data storage may include serial number and probe calibration information. In one embodiment, an electrical circuitry device 46 may comprise functionality that allows for direct measurement (via electrodes 22, 24) of the high voltage applied to the probe during fluid sample testing (see below).

In one embodiment, electrical stability probe 6A comprises a pair of terminals 50 disposed on probe tip 52, wherein each terminal 50 is in electrical communication with an electrode 22 or 24, and is in electrical communication with at least one electrical circuitry device 46. In the embodiment shown in FIG. 2, the terminals 50 are depicted as disposed in an end edge 54 of probe tip 52, however the invention is not so limited and one or both terminals 50 may be alternatively disposed. In the embodiment shown in FIG. 2, each terminal 50 is disposed recessed into probe tip 52, however the invention is not so limited and one or both terminals 50 may be alternatively disposed. In one aspect, terminals 50 are adapted and configured to be engageable with testing components, such as test plugs, thereby providing for complete functional verification of the emulsion stability tester 300, including cable 4 and probe 6A. In one embodiment, recessed terminals 50 may be fitted with replaceable fittings (plugs) (not shown) which protect the terminals 50 when they are not being utilized.

In the embodiment shown in FIG. 2, cable 4 is provided integral to probe assembly 44. In other embodiments, cable 4 may be removably attachable to probe assembly 44. In one embodiment, cable 4 is provided integral to emulsion stability meter 2, which allows for emulsion stability meter 2 to comprise a fluidly sealed component.

### Experimental Results

An experiment was conducted to compare the performance (i.e., measurable error current) of an electrical stability probe 6A with a standard electrical stability probe 6. Therein, per standard API procedures, an AC current was applied until a maximum voltage of 2000 V was achieved. As a baseline measurement, the emulsion stability meter 2 alone was tested (i.e., no electrical stability probe, no cable, and therefore no connection component there between). In one test, a standard electrical stability probe 6 was connected to the emulsion stability meter 2 via a 2.5 foot cable 4 (utilizing a connection component to connect the cable 4 to the emulsion stability meter 2). In another test, an electrical stability probe 6A was connected to the emulsion stability meter 2 via an 11 foot cable 4 (not utilizing a connection component to connect the cable 4 to the emulsion stability meter 2, but rather by directly connecting the cable 4 to the inner circuitry of the emulsion stability meter 2). The error current measured in each test is shown below:
Meter Alone: 0.5 μA at 2000 V peak
Meter with Standard Probe 6: 14.0 μA at 2000 V peak
Meter with Probe 6A: 0.5 μA at 2000 V peak
   Thus, utilization of the electrical stability probe 6A reduced the error current to an undetectable level.

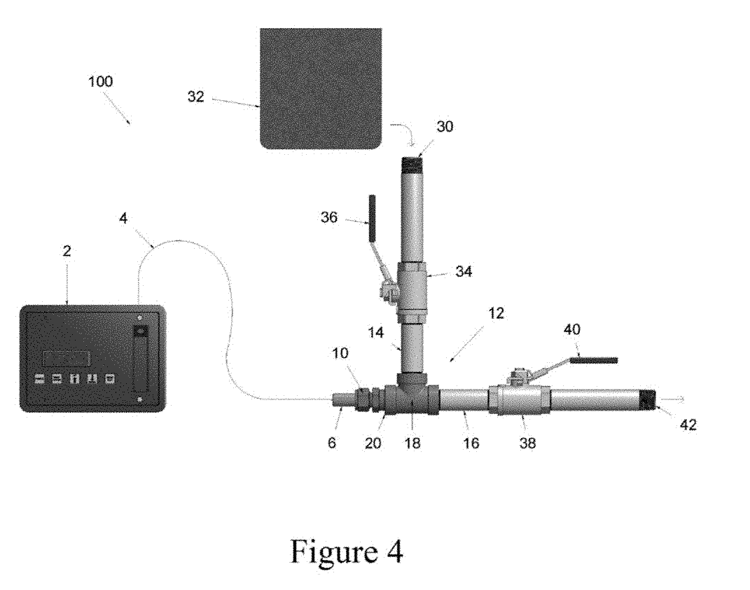

FIG. 4 depicts an embodiment of an in-line emulsion stability measurement system 100 of the present invention. In one embodiment, emulsion stability measurement system 100 comprises an emulsion stability meter 2. In one embodiment, an emulsion stability meter 2 comprises a standard device, such as the above-mentioned emulsion stability meter available from OFI Testing Equipment, Inc. of Houston, Tex., or a similar device, as is known within the art. In one embodiment, the emulsion stability meter 2 may be in informational communication with one or more processing devices (not shown), such as a computer.

In one embodiment, the emulsion stability meter 2 is connectable via cable 4 to an electrical stability probe 6 (or an embodiment of an electrical stability probe 6A as described above and depicted in FIG. 2). In one embodiment, cable 4 is adapted and configured to transmit electrical signals from emulsion stability meter 2 to electrical stability probe 6 and/or from electrical stability probe 6 to emulsion stability meter 2. In one embodiment, electrical stability probe 6 comprises a pair of electrodes 22, 24 (shown in more detail in FIG. 5) with a probe gap 8 there between, as is well known within the art.

In one embodiment, electrical stability probe 6 is combined with one or more fittings 10 whereby electrical stability probe 6 is removably attachable to a piping segment 12. Electrical stability probe 6 may be attachable to or integral with a fitting 10. In the embodiment shown in FIG. 4, piping segment 12 comprises a piping section 14 and a piping section 16 fluidly connected by a piping tee 18. The invention is not so limited, however, and other piping arrangements and configurations are contemplated. In one embodiment, piping segment 12 may comprise at least a part of a process “bypass” line, wherein a portion of the process fluid (not shown) from a process fluid source 32 is provided to electrical stability probe 6.

In one embodiment (not shown), an in-line emulsion stability measurement system 100 comprises a temperature measurement device (not shown), electrically connected to emulsion stability meter 2, which is adapted and configured to measure temperature at the surface of piping segment 12 and/or within piping segment 12. In one embodiment, the temperature measurement device (not shown) may be attachable to or integral with fitting 10. In one embodiment (not shown), the temperature measurement device (not shown) may be electrically connected to emulsion stability meter 2 at least partially via cable 4.

In the embodiment shown in FIG. 4, attachment of electrical stability probe 6 to piping segment 12 comprises connection of probe fitting 10 to piping tee 18 via piping tee 18 end connector 20. In other embodiments (not shown), electrical stability probe 6 (via fitting 10) may be alternatively attachable to piping tee 18, attachable to another variety of piping connector, or directly attachable to a piping section.

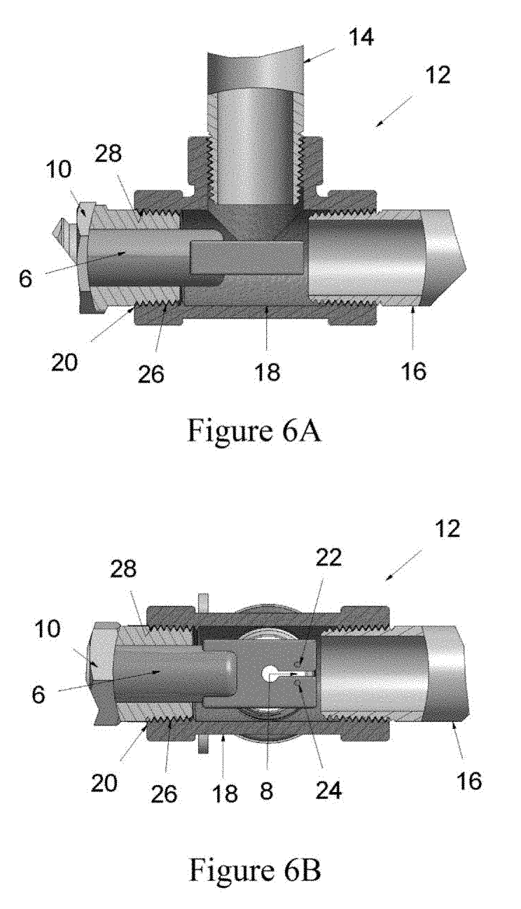

FIG. 5 depicts in greater detail a portion of standard electrical stability probe 6. In one embodiment, a fitting 10 may comprise means for attachment to piping section 12, such as, but not limited to, external threading 26 that is complementary to internal threading 28 of piping tee 18 end connector 20. (See FIG. 6A). As shown in FIG. 5, electrical stability probe 6 comprises electrodes 22 and 24 separated by probe gap 8.

FIGS. 6A and 6B depict an embodiment wherein electrical stability probe 6 is attached to piping segment 12 such that electrodes 22, 24 are disposed at least partially within the interior of piping segment 12. So disposed, process fluid (not shown) within piping segment 12 may be provided within probe gap 8; i.e., between electrodes 22, 24.

### Operation

In various embodiments, emulsion stability measurement tester 300 (FIG. 1) may be utilized to measure the emulsion stability of a process fluid sample (not shown). In one embodiment, electrical stability probe 6A is at least partially immersed in a process fluid sample (not shown) provided in a sample vessel or cell (not shown), whereby fluid is disposed within gap 8 between electrodes 22, 24. Emulsion stability measurement tester 300 is then operated to measure the emulsion stability of the process fluid (not shown). During the operation of emulsion stability measurement tester 300, an AC voltage is applied to the fluid sample (not shown) across the electrodes 22, 24, whereby induced current flow there between (current sense signal) is detected and transmitted, via electrode wires 56, 58 to at least one electrical circuitry device 46. The electrical circuitry device(s) 46 may manipulate the current sense signal, i.e., perform one or more functions on the current sense signal, as described above, and transmit a converted and/or amplified and/or filtered signal, and/or information pertaining thereto, to emulsion stability meter 2 via cable 4.

In one embodiment, additional signal(s) and/or information, relating to or comprising phenomena and/or information such as but not limited to, temperature, voltage, and stored data, may be transmitted from one or more electrical circuitry devices 46 to emulsion stability meter 2 via cable 4. In one aspect, such signals and/or information may be transmitted in a converted form or a “raw” (non-converted and/or non-amplified) form. In one embodiment, signals and/or information relating to fluid sample temperature sensed by electrode stability probe 6A during operation of emulsion stability measurement tester 300 may be transmitted to emulsion stability meter 2 via cable 4. In one embodiment, signals and/or information relating to AC voltage applied to the sample during operation of emulsion stability measurement tester 300, as sensed by electrode stability probe 6A, may be transmitted to emulsion stability meter 2 via cable 4. In one embodiment, signals and/or information relating to data stored within one or more electrical circuitry devices 46 may transmitted to emulsion stability meter 2 via cable 4 before, during, or after emulsion stability testing of a sample with the emulsion stability measurement tester 300.

In one aspect (not shown), any required cleaning of the external surfaces of electrodes 22, 24 may be accomplished by rinsing with sample fluid or some other fluid, such as by using a nozzle or other means for directing fluid flow at electrodes 22, 24, and/or by providing ultrasonic energy to electrodes 22, 24, and/or mechanically cleaning the external surfaces of electrodes 22, 24, as would be understood by one skilled in the art. In one embodiment, testing/calibration of the emulsion stability tester 300 may be performed utilizing one or more terminals 50, as would be understood by one skilled in the art.

In various embodiments, emulsion stability measurement system 100 (FIG. 4) may be utilized to measure the emulsion stability of process fluid (not shown) within piping segment 12. In one embodiment, as depicted in FIG. 4, process fluid (not shown) may be introduced to piping section 14 via pipe inlet 30. Although for illustration purposes pipe inlet 30 is depicted in FIG. 4 as not fluidly sealingly connected to a process fluid source, such as a process fluid source 32, in various embodiments (not shown) a pipe inlet 30 (or its equivalent) may be fluidly sealingly connected to one or more process fluid sources 32. In one embodiment, process fluid (not shown) is provided to piping segment 12 from process fluid source 32 via pipe inlet 30 using a pump (not shown).

In one embodiment, piping section 14 may comprise an inlet valve 34. Via manipulation of inlet valve 34, process fluid (not shown) may be provided to piping tee 18. In the embodiment depicted in FIG. 4, inlet valve 34 is manually manipulated using inlet valve handle 36. In other embodiments (not shown), inlet valve 34 may be manipulated by other means, such as remotely, by means including, but not limited to, hydraulically, pneumatically, and electrically, as would be understood by one skilled in the art.

In one embodiment, flow of process fluid (not shown) through piping section 14 to piping tee 18 provides the process fluid into contact with electrical stability probe 6. In one embodiment, process fluid (not shown) flows through piping tee 18 to piping section 16. In one embodiment, piping section 16 may comprise an outlet valve 38. In the embodiment of FIG. 4, flow of process fluid (not shown) is in the direction of the arrows (not separately labeled) depicted therein, although other flow patterns may be employed. Via manipulation of outlet valve 38, process fluid (not shown) may be flowed out of piping segment 12 via pipe outlet 42. In the embodiment depicted in FIG. 4, outlet valve 38 is manually manipulated using outlet valve handle 40. In other embodiments (not shown), outlet valve 38 may be manipulated by other means, such as remotely, by means including, but not limited to, hydraulically, pneumatically, and electrically, as would be understood by one skilled in the art.

In one embodiment, process fluid (not shown) provided within piping segment 12 is substantially sealingly contained therein by manipulation (closing) of inlet valve 34 and manipulation (closing) of outlet valve 38. In one embodiment, the outlet valve 38 is closed first, and then the inlet valve 34 is then closed. In one embodiment, once process fluid (not shown) is so contained within piping segment 12, and process fluid (not shown) is disposed between electrodes 22, 24, emulsion stability meter 2 is then operated to measure the emulsion stability of the process fluid (not shown). In one embodiment, when such measurement is accomplished, inlet valve 34 and outlet valve 38 are manipulated (opened) whereby additional process fluid (not shown) is flowed into piping segment 12 via pipe inlet 30, and process fluid (not shown) is allowed to flow out of piping section 12 via pipe outlet 42. In one embodiment (not shown), process fluid (not shown) flowing out of pipe outlet 42 may be returned to process fluid source 32, such as by piping there between.

In one embodiment, flow of process fluid (not shown) may be maintained through piping segment 12 for a period of time before inlet valve 34 and outlet valve 38 are closed to insure that residual process fluid (not shown) from a previous emulsion stability measurement sequence is “flushed” from piping segment 12. The process of flowing “fresh” process material (not shown) from process fluid source 32 into piping segment 12, “blocking in” piping segment 12 via manipulation (closing) of inlet valve 34 and outlet valve 38, may be repeated to allow for multiple sequential emulsion stability measurements of process fluid (not shown) via electrical stability probe 6 utilizing emulsion stability meter 2.

In one embodiment, any required cleaning the external surfaces of electrodes 22, 24 may be accomplished by vigorously flowing process fluid (not shown) or some other fluid (not shown) through piping segment 12, and hence through probe gap 8. In other embodiments (not shown), piping segment 12 may equipped with other means for cleaning the external surfaces of electrodes 22, 24. In various embodiments (not shown), such means may include, but are not limited to, a nozzle or other means for directing fluid flow at electrodes 22, 24; a means of providing ultrasonic energy to electrodes 22, 24; and/or a means of mechanically cleaning the external surfaces of electrodes 22, 24.

### Method

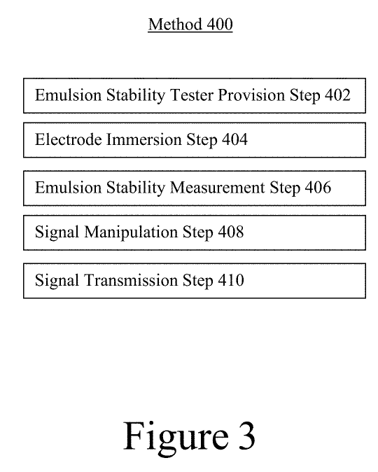

In one embodiment, an emulsion stability measurement method 400 of the present invention, as shown in FIG. 3, comprises the following steps.

An Emulsion Stability Tester Provision Step 402, comprising providing an emulsion stability tester, such as emulsion stability tester 300, comprising an emulsion stability meter, such as emulsion stability meter 2; an electrical stability probe, such as electrical stability probe 6A, comprising a pair of electrodes, such as electrodes 22, 24, a gap, such as probe gap 8, between the electrodes, and one or more electrical circuitry devices, such as electrical circuitry device 46, embedded in a probe assembly, such as probe assembly 44; and a cable, such as cable 4, electrically/informationally connecting the emulsion stability meter and the electrical stability probe.

An Electrode Immersion Step 404, comprising at least partially immersing the electrical stability probe in a fluid sample, whereby the process fluid is provided within the gap and between the electrodes.

An Emulsion Stability Measurement Step 406, comprising operating the emulsion stability tester, wherein an AC voltage is applied across the electrodes, current induced between the electrodes is detected, signals therefrom are transmitted to at least one of the one or more electrical circuitry devices, and the current sense signal is quantified.

A Current Sense Signal Manipulation Step 408, comprising utilizing at least one of the one or more electrical circuitry devices to manipulate the current sense signal.

A Signal Transmission Step 410, comprising transmitting the manipulated sensed current signal from an electrical circuitry device to the emulsion stability meter via the cable.

As would be understood by one skilled in the art, method 400 is only exemplary, and may be modified to accomplish specific results, such modifications including, but not limited to, combining, adding, deleting, re-ordering, and/or repeating one or more steps. For example, in one embodiment, steps comprising sensing voltage and/or sensing temperature, and optionally manipulating a voltage signal or a temperature signal using one or more electrical circuitry devices, and then transmitting signals (converted or raw) relating thereto from one or more electrical circuitry devices to the emulsion stability meter via the cable, are included in addition to or in lieu of one or more steps in the method. As would also be understood by one skilled in the art, while method 400 has been described as a plurality of steps, in various embodiments, two or more steps may be performed concurrently.

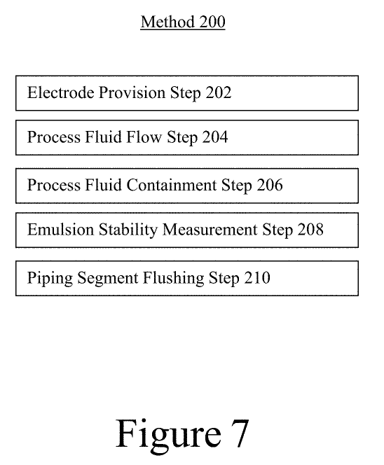

In one embodiment, an emulsion stability measurement method 200 of the present invention, as shown in FIG. 7, comprises the following steps.

An Electrode Provision Step 202, comprising providing an electrical stability probe, such as electrical stability probe 6 or 6A, in attachment with a piping segment, such as piping segment 12, wherein the electrical stability probe comprises a pair of electrodes, such as electrodes 22, 24, and a gap, such as probe gap 8, between the electrodes, and whereby the electrodes are disposed within the piping segment, and wherein the electrical stability probe is electronically connected to an emulsion stability meter, such as emulsion stability meter 2, via a cable, such as cable 4.

A Process Fluid Flow Step 204, comprising flowing a process sample from a process fluid source, such as process fluid source 32, through the piping segment, whereby the process fluid is provided within the gap and between the electrodes.

A Process Fluid Containment Step 206, comprising containing the process sample within the piping segment, by manipulating (closing) piping segment inlet and outlet valves, such as inlet valve 34 and outlet valve 38.

An Emulsion Stability Measurement Step 208, comprising operating the emulsion stability meter to determine the emulsion stability of the process sample, wherein an AC voltage is applied across the electrodes, current induced between the electrodes is detected, signals therefrom are transmitted, directly or indirectly, to the emulsion stability meter via the cable, and the current sense signal is quantified, within the electrical stability probe or the emulsion stability meter.

A Piping Segment Flushing Step 210, comprising allowing additional process fluid to flow into the piping segment and process fluid to flow out of the piping segment, by manipulating (opening) the piping segment inlet and outlet valves.

As would be understood by one skilled in the art, method 200 is only exemplary, and may be modified to accomplish specific results, such modifications including, but not limited to, combining, adding, deleting, re-ordering, and/or repeating one or more steps. As would also be understood by one skilled in the art, while method 200 has been described as a plurality of steps, in various embodiments, two or more steps may be performed concurrently.

While the present invention has been disclosed and discussed in connection with the foregoing embodiments, it will be understood that the invention is not limited to the embodiments disclosed, but is capable of numerous combinations, rearrangements, modifications, and substitutions of parts and elements without departing from the spirit and scope of the invention.

We claim:

1. An emulsion stability measurement system comprising:
an electrical stability probe;
wherein:
said electrical stability probe comprises two opposing electrodes separated by a gap there between;
in response to a voltage applied to across said gap, said electrical stability probe senses electrical signals when a fluid sample is disposed within said gap;
said electrical stability probe comprises at least one electrical circuitry device embedded in a probe assembly;
at least one said electrical circuitry device functions to manipulate said electrical signals sensed by said electrical stability probe; and
at least one said electrical circuitry device functions to transmit information and/or electrical signals from said electrical stability probe to an emulsion stability meter device via a cable.

2. The emulsion stability measurement system of claim 1, wherein at least one said electrical circuitry device comprises a printed circuit board.

3. The emulsion stability measurement system of claim 1, wherein manipulation of said electrical signals sensed by said electrical stability probe comprises converting said sensed electrical signals to a differential analog signal and/or digital signal.

4. The emulsion stability measurement system of claim 1, wherein manipulation of said electrical signals sensed by said electrical stability probe comprises amplifying said sensed electrical signals.

5. The emulsion stability measurement system of claim 1, wherein said at least one said electrical circuitry device comprises functionality selected from the group consisting of:
temperature measurement;
data storage;
measurement of a voltage applied across said gap;
measurement of current flowing through said gap; and
combinations thereof.

6. The emulsion stability measurement system of claim 1, wherein said electrical stability probe comprises a plurality of terminals, each in electrical communication with one said electrode and at least one said electrical circuitry device, wherein said terminals allow for functional verification of one or more components of said emulsion stability measurement system.

7. A method for emulsion stability measurement comprising:
providing an emulsion stability measurement system comprising an electrical stability probe, wherein:
said electrical stability probe comprises two opposing electrodes separated by a gap there between;
in response to a voltage applied to across said gap, said electrical stability probe senses electrical signals when a fluid sample is disposed within said gap;
said electrical stability probe comprises at least one electrical circuitry device embedded in a probe assembly;
at least one said electrical circuitry device functions to manipulate said electrical signals sensed by said electrical stability probe; and
at least one said electrical circuitry device functions to transmit information and/or electrical signals from said electrical stability probe to an emulsion stability meter device via a cable;
immersing said electrical stability probe at least partially into a fluid sample, whereby at least a portion of said fluid sample is disposed within said gap and between said electrodes;
applying an AC voltage across said electrodes, thereby inducing a current there between;
sensing said current using said electrodes and transmitting electrical signals therefrom to at least one said electrical circuitry device;
utilizing at least one said electrical circuitry device to manipulate one or more electrical signals received thereby; and
transmitting at least one said manipulated signal from at least one said electrical circuitry device to said emulsion stability meter device via said cable.

8. The method of claim 7, wherein manipulation of said electrical signals sensed by said electrical stability probe comprises at least one process selected from the group consisting of:
converting said sensed electrical signals to a form comprising a differential analog signal and/or digital signal;
filtering said sensed electrical signal, said differential analog signal, and/or said digital signal;
amplifying said sensed electrical signal, said differential analog signal, and/or said digital signal; and
combinations thereof.

9. The method of claim 7, wherein at least one manipulated signal can be transmitted to said emulsion stability meter device via said cable with less signal current error than said signal in a non-manipulated form.

10. An in-line emulsion stability measurement system comprising:
an electrical stability probe;
wherein:
said electrical stability probe comprises two opposing electrodes separated by a gap there between;
said electrical stability probe communicates electronically with an emulsion stability meter device via a cable;
said electrical stability probe is equipped with a fitting that allows for attachment of said electrical stability probe to a process piping segment, whereby said electrodes and said gap are at least partially disposed within the interior of said process piping segment;
said process piping segment allows for fluid communication between said process piping segment and an industrial process apparatus, whereby process fluid from said industrial process apparatus is introducible to said interior of said process piping segment; and
contact between said process fluid and said electrodes, within said gap, allows for in-line measurement of emulsion stability of said process fluid within said process piping segment, via operation of said emulsion stability meter device.

11. The emulsion stability measurement system of claim 10, wherein:
said electrical stability probe comprises at least one electrical circuitry device embedded in a probe assembly;
at least one said electrical circuitry device functions to manipulate said electrical signals sensed by said electrical stability probe; and
at least one said electrical circuitry device functions to transmit information and/or electrical signals from said electrical stability probe to an emulsion stability meter device via a cable.

12. The emulsion stability measurement system of claim 10, wherein a temperature measurement device that measures temperature within or proximate said process fluid is in informational communication with said emulsion stability meter device.

13. The emulsion stability measurement system of claim 11, wherein at least one said electrical circuitry device comprises a printed circuit board.

14. The emulsion stability measurement system of claim 11, wherein manipulation of said electrical signals sensed by said electrical stability probe comprises converting said sensed electrical signals to a differential analog signal and/or digital signal.

15. A method for in-line emulsion stability measurement comprising:
providing an in-line emulsion stability measurement system comprising an electrical stability probe;
wherein:
said electrical stability probe is in electronic communication with an emulsion stability meter device via a cable;
said electrical stability probe comprises two opposing electrodes separated by a gap there between;
said electrical stability probe is equipped with a fitting that allows for attachment of said electrical stability probe to a process piping segment;
said process piping segment allows for fluid communication between said process piping segment and an industrial process apparatus, whereby process fluid from said industrial process apparatus is introducible to said interior of said process piping segment; and
contact between said process fluid and said electrodes, within said gap, allows for in-line measurement of emulsion stability of said process fluid within said process piping segment, via operation of said emulsion stability meter device;
attaching said electrical stability probe, via said fitting, to said process piping segment, whereby said electrodes and said gap are at least partially disposed within the interior of said process piping segment;
introducing said process fluid into said interior of said of said process piping segment, whereby said process fluid contacts said electrodes within said gap; and
measuring said emulsion stability of said process fluid within said process piping segment, via operation of said emulsion stability meter device.

16. The method of claim 15 comprising measuring temperature within or proximate said process fluid.

17. The method of claim 15, wherein:
said electrical stability probe comprises at least one electrical circuitry device embedded in a probe assembly;
at least one said electrical circuitry device functions to manipulate said electrical signals sensed by said electrical stability probe; and
at least one said electrical circuitry device functions to transmit information and/or electrical signals from said electrical stability probe to an emulsion stability meter device via a cable.

18. The method of claim 17, wherein at least one said electrical circuitry device comprises a printed circuit board.

19. The method of claim 15, wherein manipulation of said electrical signals sensed by said electrical stability probe comprises at least one process selected from the group consisting of:
converting said sensed electrical signals to a form comprising a differential analog signal and/or digital signal;
filtering said sensed electrical signal, said differential analog signal, and/or said digital signal;
amplifying said sensed electrical signals, said differential analog signal, and/or said digital signal; and
combinations thereof.

20. The method of claim 17, wherein at least one manipulated signal can be transmitted to said emulsion stability meter device via said cable with less current error than said signal in a non-manipulated form.
